(19) United States
(12) Patent Application Publication
(10) Pub. No.: US 2004/0265830 A1
(43) Pub. Date: Dec. 30, 2004

(54) Methods for identifying differentially expressed genes by multivariate analysis of microaaray data

(76) Inventors: Aniko Szabo, Layton, UT (US); Alexander Tsodikov, Sacramento, CA (US); Andrei Yakovlev, Rochester, NY (US); Kenneth Boucher, Salt Lake City, UT (US); David A. Jones, Salt Lake City, UT (US); William Carroll, Irvington, NY (US)

Correspondence Address: HELLER EHRMAN WHITE & MCAULIFFE LLP, 1666 K STREET,NW, SUITE 300, WASHINGTON, DC, 20006, US

(21) Appl. No.: 10/492,599

(22) PCT Filed: Oct. 17, 2002
(86) PCT No.: PCT/US02/33115

Related U.S. Application Data
(60) Provisional application No. 60/329,531, filed on Oct. 17, 2001.

(51) Int. Cl. C12Q 1/68

(52) U.S. Cl. 435/6

(57) ABSTRACT

The present invention applies pattern recognition and multidimensional classification in statistical microarray data analysis. There are provided methods for identifying a subset of genes that are differentially expressed under a given biological state or at a given biological locale of interest by determining an advantageously large probability distance between the random vectors representing expression levels of the genes of interest. The invention also provides a cross-validated random search mechanism for a subset of genes based on various probability distances between two random vectors.

### BACKGROUND OF THE INVENTION

1. Field of the Invention

The present invention relates in general to statistical analysis of microarray data generated from arrays, and in particular nucleotide arrays. Specifically, the present invention provides improved methods for identification of differentially expressed genes by microarray data analysis. More specifically, the present invention provides methods for determining an advantageously large probability distance between certain random vectors thereby identifying a subset of genes that are differentially expressed under a given biological state or at a given biological locale of interest.

2. Description of the Related Art

The emergence and evolution of microarray technologies in recent years coincides with the progress and completion of the sequencing of human genome and the genomes of a number of other species. Today, researchers are able to simultaneously monitor expression patterns of thousands of genes, using oligonucleotide and DNA probes designed and/or selected based on the newly available genomic sequence information. Proteome chips are also used. See Zhu et al., Science 293:2101 (2001). Large scale and high throughput expression studies provide better understanding of functions of the genes of interest, interactions of the encoded proteins, and the biological pathways or processes involving these genes. Ultimately, the knowledge of changes of gene expression and information on perturbations to gene function and operation of pathways under different biological or pathological conditions is vitally important and directly contributes to research on the mechanisms of life and effective diagnostics and therapeutics.

To fully realize the power and promise of microarray technologies, however, effective methods are needed to accurately interpret microarray data and thereby provide useful information that is of biological, medical, or pharmaceutical relevance. For example, methods to identify distinct patterns and changes of patterns, to group and classify genes, and to determine the differences among individual genes or subsets of genes are important in identifying differentially expressed genes in certain tissues or cells or at certain biological or pathological states.

Little work has been done to develop statistically sound methods for identifying differentially expressed genes. One approach is to use the is logarithm of the ratio of the observed fluorescence signals from different samples and consider any gene with the ratio above a fixed value (e.g., 2 or 3) to be differentially expressed. See DeRisi J. et al., Nature Genetics 1996 14(4):457-460; Schena M. et al., Science 270 1995 5235:467-470. Others have looked to the application of statistical classification or pattern recognition in search for differentially expressed genes.

Statistical pattern recognition can be formulated within the framework of discriminant analysis. Each pattern is considered as an entity that belongs to one of a number of predefined classes or groups of patterns (tissues or states, for example) and can be represented by a vector of feature variables. In the context of microarray data analysis, a set of microarray data (e.g., signals of expression levels) on a distinct set of genes can be represented by a random vector. Certain rules for initial feature vector selection in pattern recognition have been proposed. For example, van der Laan and Bryan (Public Health series 86, Univ. of Cal., Berkeley 2000) suggested the following procedure: (1) select those genes which are at least m-fold differentially expressed (m is predetermined by the user) with respect to the marginal mean level of expression in the two states (tissues) under comparison; (2) estimate a correlation-distance matrix for these differentially expressed genes; (3) apply a clustering algorithm to this distance-matrix; and optionally (4) only include those genes in the target subset that are closest to the cluster centers. Newton et al. (J. of Comp. Biol. 2000 8(1): 37-52) obtained an empirical Bayes estimator of the fold change in gene expression that turned out to be different from the simple ratio. Kerr et al. (J. of Comp. Biol. 2000 7(6):819-837) proposed to use ANOVA for the log-intensities to combine the adjustment step with the identification of differential expression. Some researchers also raised the issue of experimental design for microarray studies and pointed out confounding effects in typical experimental setups. Many felt that better ways of measuring differences in the expression of a gene or a set of genes in different tissues or states remained to be explored. See Id.

One characteristic of the typical classification methods applied to gene expression data (See, e.g., Dudoit et al., 2000, Tech. Rep. 576, Univ. Of Cal., Berkeley) is the univariate nature of the decision to include a particular gene in the initial feature set. The complex interaction pattern of gene functions makes it unlikely that the contribution of a gene to the between-tissue (or state) difference can be evaluated only marginally. Methods that use multivariate information at every step are needed to utilize the information hidden in gene interactions and hence to increase the power of classification rules.

### SUMMARY OF THE INVENTION

It is therefore an object of this invention to provide methods for analyzing gene expression data generated from probe arrays, taking into account the multidimensional structure of microarray data. Particularly, it is an object of this invention to apply multivariate analysis methods to microarray gene expression data thereby identifying subsets of genes that are differentially expressed.

In accordance with the present invention, there is provided a method for identifying a set of genes from a multiplicity of genes whose expression levels at two states, in two tissues, or in two types of cells, or any combination thereof, are measured in replicates using one or more probe arrays, thereby generating a plurality of independent measurements of the expression levels, wherein the set is no more larger than the plurality, which method comprises: constructing two random vectors, each corresponding to one of the two states and comprising the expression levels of a group of genes, wherein the group is a random subset of the multiplicity; identifying a probability distance formula; calculating probability distance(s) between the two random vectors based on the probability distance formula; and determining an advantageously large probability distance between the two random vectors; wherein the group of genes which constitute the two random vectors giving rise to the advantageously large probability distance is the set of genes identified.

According to the present invention, in certain embodiments, the states may be biological states, physiological states, pathological states, and diagnostic or prognostic states. For examples, the states may be, inter alia, normal and abnormal states, normal and diseased states, resting and activated states, stimulated and unstimulated states, etc. In other embodiments, the tissues may be, inter alia, normal lung tissues, abnormal lung tissues or cancer lung tissues, normal heart tissues, pathological heart tissues, normal and abnormal colon tissues, normal and abnormal renal tissues, normal and abnormal prostate tissues, and normal and abnormal breast tissues. In yet other embodiments, the types of cells may be normal lung cells, abnormal lung cells, cancer lung cells, normal heart cells, pathological heart cells, normal and abnormal colon cells, normal and abnormal renal cells, normal and abnormal prostate cells, and normal and abnormal breast cells. In still other embodiments, the types of cells may be cultured cells and primary cells isolated from an organism. The skilled artisan will recognize that the methods described herein are applicable to comparative analysis of essentially any types of array data.

According to certain embodiments of the present invention, the advantageously large distance is a maximal probability distance taken over the plurality of independent measurements. In other embodiments, the arrays may be arrays of probe molecules, for example, nucleotide arrays containing spotted full-length or partial cDNA sequences and/or arrays of in situ synthesized oligonucleotides.

According to certain embodiments of this invention, the distance between vectors may be the Mahalanobis distance or the Bhattacharya distance. According to another embodiment, the probability distance formula is

N(λ,v)=2∫_{R}_{^{d}}∫_{R}_{^{d}}L(x,y)dμ(x)dv(y)−∫_{R}_{^{d}}∫_{R}_{^{d}}L(x,y)dμ(x)dμ(y)−∫_{R}_{^{d}}∫_{R}_{^{d}}L(x,y)dv(x)dv(y)

where μ and v are two probability measures defined on the Euclidean space, and L(x,y) is a strictly negative definite kernel. According to yet another embodiment, the negative definite kernel is combined with the Euclidean distance between x and y to form a composite kernel function. According to still another embodiment, the negative definite kernel is based on the correlation coefficient and is capable of detecting differences in correlation between the two random vectors.

According to other embodiments of this invention, the expression levels are adjusted to their corresponding fractional ranks as compared to one another and thereafter used to construct the vectors. In yet other embodiments, each of the expression levels is adjusted to a corresponding categorical descriptor of the extent of over or under expression and thereafter used to construct said vectors.

### BRIEF DESCRIPTION OF DRAWINGS

FIG. 1 depicts the steps of cross-validated search for subsets of genes based on calculation of a probability distance between vectors according to certain embodiments of the invention.

FIG. 2 depicts rank adjusted expression levels of genes in the ALL/AML data set; the upper panel shows the ALL samples, the lower panel the AML samples. The set of genes listed are identified by cross-validated search for a maximized distance estimate. The identities of the genes are: 2288, D component of complement (adipsin); 2335, immunoglobulin-associated beta (B29); 6378, NF-IL6-beta protein mRNA; 1882, cystatin C; 6200, interleukin 8 (IL8) gene; 6218, elastase 2, neutrophil; 4680, TCL1 gene (T cell leukemia); 3252, glutathione S-transferase; 6219, neutrophil elastase gene, exon 5; and 6308, GRO2 oncogene.

### DETAIL DESCRIPTIONS OF DISCLOSURE

Definition

As used herein, the term “microarray” refers to arrays or probe molecules that can be used to detect analyte molecules, for instance to measure gene expression. Such microarrays may be nucleotide arrays or peptide or protein arrays; “array,” “slide,” and “chip” are used interchangeably in this disclosure. Various kinds of arrays are made in research and manufacturing facilities worldwide, some of which are available commercially. There are, for example, two main kinds of nucleotide arrays that differ in the manner in which the nucleic acid materials are placed onto the array substrate: spotted arrays and in situ synthesized arrays. One of the most widely used oligonucleotide arrays is GeneChip™ made by Affymetrix, Inc. The oligonucleotide probes that are 20- or 25-base long are synthesized in silico on the array substrate. These arrays tend to achieve high densities (e.g., more than 40,000 genes per cm²). The spotted arrays, on the other hand, tend to have lower densities, but the probes, typically partial cDNA molecules, usually are much longer than 20- or 25-mers. A representative type of spotted cDNA array is LifeArray made by Incyte Genomics. Pre-synthesized and amplified cDNA sequences are attached to the substrate of these kinds of arrays. Protein and peptide arrays also are known. See Zhu et al., supra.

Microarray data, as used herein, encompasses any data generated using various probe arrays, including but not limited to the nucleotide arrays described above. Typical microarray data include collections of gene expression levels measured using nucleotide arrays on biological samples of different biological states and origins. The methods of the present invention may be employed to analyze any microarray data; irrespective of the particular microarray platform from which the data are generated.

Gene expression, as used herein, refers to the transcription of DNA sequences, which encode certain proteins or regulatory functions, into RNA molecules. The expression level of a given gene measured at the nucleotide is level refers to the amount of RNA transcribed from the gene measured on a relevant or absolute quantitative scale. The expression level of a given gene measured at the protein level refers to the amount of protein translated from the transcribed RNA measured on a relevant or absolute quantitative scale. The measurement can be, for example, an optic density value of a fluorescent or radioactive signal, on a blot or a microarray image. Differential expression, as used herein, means that the expression levels of certain genes, as measured at the nucleotide or protein level, are different in different states, tissues, or type of cells, according to a predetermined standard. Such standard maybe determined based on the context of the expression experiments, the biological properties of the genes under study, and/or certain statistical significance criteria.

The terms “vector,” “probability distance,” “distance,” “the Mahalanobis distance,” and “the Euclidean distance” are to be understood consistently with their typical meanings established in the relevant art, i.e. the art of mathematics, statistics, and any area related thereto. For example, a set of microarray data on p distinct genes represents a random vector X=X₁, . . . , Xₚ with mutually dependent components.

Searching for the Initial Feature Vector

Suppose, for n independent observations of gene expression in a given state of the biological system under study, the same genes are expressed at certain levels subject to random variation in expression. This set of observations forms an observation matrix of dimension n×p, where p is the total number of genes. The initial step of multidimensional classification is to reduce the full feature vector represented by the data on expression of all genes. Most of the nucleotides spotted on the array represent genes that are not involved in the processes that distinguish the two samples under comparison. As mentioned above, current methods for determining differentially expressed genes are based on univariate choices. Those approaches ignore the correlation information contained in the data and thus may limit the power of classification rules. In addition, the selection of the feature set is not closely related to the classification of unknown entities in those methods. Thus, while the gene selection process may select significant genes in the sense of marginal differential expression, they may not be the best choice as a feature set for the classification method.

It is therefore desirable to search for a subset (cluster) of genes that in some sense differs the most between two tissues or states thereby developing a classification rule based on the same notion of difference. To this end, the present invention provides a pertinent probability distance between two subsets of genes. This probability distance is a probability distance (metric) whose empirical counterpart may combine information from different chips or arrays; it may accommodate rank data as well as categorical data, and hence does not necessarily assume normality. The computation of the distance should not be too time consuming. Because the calculation of the distance is based on an entire gene set rather than separately on each gene, the multidimensional information on gene expression are better utilized and accounted for. A gene set or cluster of size one may be a special case in applying this probability distance; thus, this approach also may improve univariate procedures of variable selection.

Differential Expression of Subsets of Genes Determined by Calculation of a Probability Distance

As discussed above, multidimensional information contained in sample observations are often disregarded in the feature prediction and classification of microarray data. See, e.g., Hastie et al., Genome Biology 2000 1(2): 0002.1-0003.21. Yet, a high correlation between expression levels for individual genes and the large cluster sizes compared to the number of independent replicates hampers the use of two-sample statistical tests. In discriminant analysis settings, the Mahalanobis distance may be used as the measure of distance between two groups when the feature variables are continuous. The distance is defined as follows: if the feature vector Y is drawn from a two-variate distribution with means m₁ and m₂, and common covariance matrix S, then

R_{Mah}²=(m₁−m₂)′S⁻¹(m₁−m₂).

To ensure the nonsingularity of the matrix S estimated from sample observations one should impose the constraint: n>d, where n is the sample size, d≦p is the number of genes in the target subset. The same may apply to the Chernoff distance in the multivariate normal case. In addition, empirical counterparts of these distances in actual data analyses, as well as those based on kernel estimates of multivariate distributions may be used. And, different versions of Mahalanobis distance may also be used in various embodiments of this invention, such as the ones that are derived from some functions of trimmed or Winsorized variances. See, e.g., Gnanadesikan R., Methods of Statistical Data Analysis of Multivariate Observations, Wiley, 1997, 2nd ed. The computation of these distances may, in some cases, be sensitive to experimental noise or require significant computational power.

According to one embodiment, the present invention provides another probability distance and its nonparametric estimate to measure differential expression between subsets of genes. Let μ and ν be two probability measures defined on the Euclidean space. Let L(x,y) be a strictly negative definite kernel, that is Σ_{l,f=1}^{s}L(xᵢ,xⱼ)hᵢhⱼ≦0 for any x₁, . . . ,xₛ and h₁, . . . ,hₛ, Σ_{f=1}^{s}hᵢ==0 with equality if and only if all hᵢ=0. Introduce the following expression

N(μ,ν)=2∫_{R}_{^{d}}∫_{R}_{^{d}}L(x,y)dμ(x)dν(y)−∫_{R}_{^{d}}∫_{R}_{^{d}}L(x,y)dμ(x)dμ)−∫_{R}_{^{d}}∫_{R}_{^{d}}L(x,y)dν(x)dν(y)

It can be shown that N(μ, ν) is a metric in the space of all probability measures on ∇^{d}. See Zinger A A. et al., Stability Problems for Stochastic Models VNIISI, Moscow 1989, p 47-57).

Consider two independent samples, consisting of n₁ and n₂ observations respectively, represented by the d-dimensional vectors x₁, . . . , xₙ₁ and y₁, yₙ₂, and introduce an empirical counterpart of N(μ, ν) as follows

$$\begin{matrix}\hat{N} = N ( {μ}_{n1} , {v}_{n2} ) \\ = \frac{1}{{n}_{1} {n}_{2}} {\sum }_{i = 1}^{n1} {\sum }_{j = 1}^{n2} 2 L ( {x}_{i} , {y}_{j} ) - \\ \frac{1}{{n}_{1}^{2}} {\sum }_{i = 1}^{n1} {\sum }_{j = 1}^{n1} L ( {x}_{i} , {x}_{j} ) - \frac{1}{{n}_{2}^{2}} {\sum }_{i = 1}^{n2} {\sum }_{j = 1}^{n2} L ( {y}_{i} , {y}_{j} )\end{matrix}$$

When using the distance N(μ, ν) one needs to choose a pertinent kernel function L. One choice is the Euclidean distance between ranks. Yet another possibility is to use the Mahalanobis distance R_{Mah} assuming the two groups (classes) under comparison have the same positive definite correlation matrix. This invention, in another embodiment, provides an alternative class of kernel functions that may be used to measure pairwise gene interaction.

Let x and y denote observations in two samples on a gene set and x^{r} and y^{r} denote the corresponding rank-adjusted observations. Consider either of these observations to be points in Euclidean space. Let S be a measurable subset of ∇^{d}. Define L_{S} by the rule L_{S}(x,y)=0 if both xεS and yεS and L_{S}(x,y)=1 otherwise. L_{S} is a negative definite kernel. Suppose, xᵢεS, 1≦i≦r, and xᵢ∉S, r+1≦i≦s, then one would have

Σ_{i,j=1}^{s}(1−L_{S}(xᵢ,yⱼ))hᵢhⱼ=Σ_{i,j=1}^{r}hᵢhⱼ=(Σ_{i,j=1}^{r}hᵢ)²≧0. Thus (1−L_{S}) is a positive definite kernel.

More generally, let ƒ(x) be a function from a space to the interval [0,1], and define L_{ƒ}(x,y)=max(ƒ(x),ƒy)), then L_{ƒ} is a negative definite kernel. Also, if one defines gₐ(x,y)=0 provides both ƒ(x)>a and ƒ(y)>a and gₐ(x,y)=1 otherwise, then, from the previous paragraph, gₐ is a negative definite kernel. It follows from the equality L_{ƒ}(x,y)=∫₀¹gₐ(x,y)da that L_{ƒ} is negative definite. Since a negative definite kernel is unaffected by an arbitrary additive shift, it is clear that L_{ƒ}(x,y)=max(ƒ(x),ƒ(y)) will be a negative definite kernel for any bounded function ƒ.

If wᵢ are positive weights and ƒᵢ, 1≦i≦d, are functions from to [0,1], then L=Σᵢ₌₁^{d}wᵢL_{ƒ}_{ᵢ} is also a negative definite kernel. From the foregoing derivations, one would also have: if {ƒᵢ} separates points, that is, ƒᵢ(x)=ƒᵢ(y) for all i implies x=y, then L is strictly negative definite.

Negative definite kernels of the type described above may be combined with the usual Euclidean distance to form composite kernel functions. For example, define a region function R_{q}(u,v)=q└qu┘+└qv┘ (here └•┘ denotes the floor function, its value is the largest integer not exceeding the argument and q≧2 is an integer parameter). This function is constant on each of the q² obtained by dividing the sides of the (0,1)² into q equal segments. Then the following kernels on the ranked data may be defined:

$${L}_{1} ( {x}^{r} , {y}^{r} ) = \sqrt{{\sum }_{g ∈ S} {( {x}_{g}^{r} - {y}_{g}^{r} )}^{2}} , \\ {L}_{2} ( {x}^{r} , {y}^{r} ) = {w}_{1} {L}_{1} ( {x}^{r} , {y}^{r} ) + {w}_{2} {\sum }_{( {g}_{1} , {g}_{2} ) ∈ {S}^{2}} ( 1 - I \{ {R}_{q} ( {x}_{{g}_{1}}^{r} , {x}_{{g}_{2}}^{r} ) = {R}_{q} ( {y}_{{g}_{1}}^{r} , {y}_{{g}_{2}}^{r} ) \} ) ,$$

where I is the indicator function. Then L₁ is the standard Euclidean distance and L₂ falls into the class described above. We choose the weights w₁ and w₂ to balance the two components of L₂ with respect to their maximum values:

$${w}_{1} = 1 / {d}_{max} and {w}_{2} = 1 / ( \begin{matrix}{d}_{max} \\ 2\end{matrix} ) ,$$

where dₘₐₓ, is the maximum subset dimension under consideration. The second component of the kernel will be insensitive to perturbation, yet pick up sets of genes that have similar expression levels across samples in one tissue and different expression patterns in the two tissues.

In another alternative embodiment, a function L_{ƒ} is based on the correlation coefficient. Let xₙ and yₙ denote normalized data such that the tissue-specific sample mean and variance are zero and one respectively. For each pair of genes g₁ and g₂, consider the function ƒ_{g}_{₁}_{,g}_{₂} (xⁿ)=x_{g}_{₁}ⁿx_{g}_{₂}ⁿ. The corresponding negative definite kernel L_{g1,g2} will detect differences in correlation between the two tissues. For example, if the expressions of g₁ and g₂ have correlation coefficient ρ in one tissue and are uncorrelated in the other, it follows from 2 max(ρ,0)−max(ρ,ρ)−max(0,0)=|ρ| that the corresponding distance between the tissues will be approximately equal to |ρ|.

A negative definite kernel may, in this embodiment, be defined as:

$${L}_{3} ( x , y ) = {w}_{1} {L}_{1} ( x , y ) + {w}_{2} {\sum }_{( {g}_{1} , {g}_{2} ) ∈ {S}^{2}} {{L}_{{g}_{1} , g}}_{2} ( x , y )$$

The weights w1 and w₂ may be chosen to balance the contribution of the two components. A distance based on L₃ will tend to pick up sets of genes with separated means and differences in correlation in the two samples.

Random Search for Differentially Expressed Subsets of Genes

As discussed above, it is desirable to select the best subset of genes in accordance with a predetermined selection criterion, avoiding using too many feature variables. Selecting too many feature variables can deteriorate the performance of a discriminant rule. The rate of misallocation of unclassified entities is one criterion that is typically used for the choice of feature variables. Several useful error-based procedures have been proposed under the assumption of the homoscedastic normal model. See, e.g., McLachlan G J., Discriminant Analysis and Statistical Pattern Recognition Wiley, 1992. These procedures are formulated in the form of a statistical test with an adjustment for multiple testing. With stepwise selection procedures, as noted by McKay and Campbell (Br. J. Math. Statist. Psychol. 1982, 35: 141), the tests are not independent and it is difficult to design a theoretically sound adjustment to control the simultaneous significance level for the sequence of tests.

The present invention provides methods, in various embodiments, for selecting a reduced feature vector and testing for differentially expressed subsets of genes. In practical settings, it may be challenging to examine all is possible subsets in order to find the one for which the distance between two groups of entities is maximal. One may resort to the branch-and-bound algorithm when the size of a target subset is small See, Fukunaga K., Introduction to Statistical Pattern Recognition (Academic Press, London, 1990), 2nd. ed. The algorithm finds a maximum and it is generally more efficient than the straightforward checking of all possibilities. The branch-and-bound method works best when the initial vector is close to the optimal and, when the intrinsic dimension of the feature space is small. See Id. Fukunaga provides empirical evidence that the method works well on uniformly distributed data when the intrinsic dimension is two and poorly when the intrinsic dimension is eight.

Since the number of possible subsets exponentially increases with the total number of genes, stepwise and/or iterative procedures become necessary aid to variable selection. According to one embodiment, the present invention provides a random search method for finding a cluster or subset of k genes with the largest distance between the two classes (tissues or states). Such method is rather insensitive to irregularities of the underlying optimization problem and to the presence of noise in the objective function. It is especially advantageous in dealing with computational complexities for relatively large subsets of genes. Briefly, the method comprises the following steps: (i) randomly select k genes to form the initial approximation and calculate the distance between the two classes for this cluster/subset; (ii) replace at random one gene from the current cluster/subset by a gene from outside the cluster/subset and calculate the distance for this new cluster/subset; (iii) if the distance for the new cluster is larger than for the original cluster/subset, keep the change, otherwise revert to the previous cluster/subset; and (iv) repeat steps ii and iii until convergence.

In another embodiment, the present invention provides an alternative random search method to reduce selection bias. Cross-validation is used in this method to eliminate or alleviates the problem of overfitting, i.e., finding overly specific patterns that do not extend to new samples. Briefly, the method comprises the following steps: (i) randomly divide the data into v groups of nearly equal size; (ii) drop one of the parts and find the optimal (in accordance with the predetermined criterion) subset of genes using only the data from v-1 groups; (iii) repeat step ii in succession for each of the groups and obtain v-optimal sets; and (iv) combine these sets by selecting the genes with the highest frequencies of occurrence. A detailed example of cross-validated search method is discussed infra in Example 3.

Categorical and Fractional Rank Adjustments of Microarray Gene Expression Data

Effective microarray data analysis often requires preprocessing of raw data from array or chip images. Various background reduction, normalization, and other adjustment procedures may be used. Such data adjustment is transforms the measurements of gene expression such that they are placed on the same scale. Statistical tests can then be applied to the transformed signals, a surrogate of ideal measurements. Data adjustments may be formulated based on specific models of gene expression signals. According to one embodiment of the invention, the actual expression signals are replaced with their fractional rank (the rank divided by the total number of genes) within the array:

Xᵢⱼ^{(r)}=(rankⱼXᵢⱼ)/p,Yᵢⱼ^{(r)}=(rankⱼYᵢⱼ)/p,

where rankⱼ uᵢⱼ is the place (counted from the left) of uₖj in the sequence uᵢⱼ; i=1, . . . , p arranged in decreasing order for each j.

In many practical situations, this adjustment restores the correct ordering of observations, i.e., gene expression levels, in the presence of experimental noise of a fairly general structure. This adjustment is also resistant to outliers. However, there may be a loss of information resulting from this adjustment in some situations. For example, the expression of a given gene may change significantly with its rank remaining unchanged. Conversely, the rank of a given gene may change (because of changes in expression of other genes) while there is no change in its own expression level. Moreover, identical distribution of ranks in two tissues does not necessarily imply identical distribution of the corresponding vectors of expression signals. Also, if the components of some subvector of gene expression signals behave as independent and identically distributed random variables, then the ranks of all the genes included in this subvector are equally likely. Thus, it would require large sample sizes to make statistical inference from ranked observations on such a subvector. Nevertheless, this scenario is rather rare in microarray data analysis. Rank based data adjustment is a useful aid according to this invention before subjecting data to the analysis and search methods discussed above.

According to another embodiment of this invention, microarray data is subject to a categorical adjustment before being analyzed. Particularly, a scatter plot of expression measurements is used. A measurement of fluorescent intensity in two channels (x=Green and y=Red) gives a point (x, y) on the plane. A set of all such points for the genes associated with a given slide forms a scatter plot. Ideally, non-differentially expressed genes would preserve a constant Green/Red ratio of 1, the corresponding (x, y) points building a line on the plane. A differentially expressed gene would ideally show a different ratio, the corresponding points being away from the line. However, for a number of reasons the picture is more complex: (i) additive background effect provides for a non-zero intercept of the line; (ii) due to measurement errors and random nature of gene expression, the points corresponding to non-differentially expressed genes are scattered considerably around the line; and (iii) a strong slide-specific effect makes the scale and the scatter plot pattern variable from slide to slide.

Suppose a sample of x and y values is drawn from a system (vector) of dependent random variables with an unknown dependency structure. The set of values {(xᵢ, yᵢ)}ᵢ₌₁^{p} contains an unknown fraction of outliers that are not expected to follow the line. Also, both x and y are subject to measurement error. In a situation where both x and y are measured with error, a linear structural relationship is nonidentifiable without additional constraints. Even in the simplest case of independent measurements, a least squares line for the model

Xᵢ=Uᵢ+δᵢ Yᵢ=Vᵢ+εᵢ

where δ and ε are measurement errors, and V=a+bU, underestimates the slope b of the latent structural relationship.

For this reason, an ad hoc method is used in this embodiment of the invention to define a reference line for the scatter plot: Once the reference line is determined, it is rotated rigidly to coincide with the x-axis and all p points of the scatter plot are projected on the line by the closest point projection. The coordinate system is changed from (x, y) to (t, d), where d is a signed (directed) distance from the point (x, y) to its projection, and t is a similar distance from the projection to the minimal projection on the reference line. The signed distance d quantifies an instance of differential expression for a particular gene on the slide. Points above the line bear a positive d indicating potential overexpression, while negative d is a sign of potential underexpression.

The distribution of d for genes in some small interval [t−Δ, t+Δ] appears to be a function of t, indicating that genes with different order of absolute expression cannot be measured on the same d-scale. Therefore, d is not directly used as a surrogate of differential expression. A summary measure of differential expression can be constructed by ranking genes with respect to the directional distance d adjusted for the surrogate of absolute expression signal t. To categorize differential expression, define a cross section layer W₁⁺={0<d<∞,t−Δ(t)<t<t+Δ(t)}, where Δ(t) is a bandwidth. Similarly, W₁⁻={−∞<d<0, t−Δ(t)<t<t+Δ(t)}. Define a set of cutpoints αⱼ, j=0, . . . ,k+1 that break the interval of total probability [0,1] down into k+1 subintervals. By definition α₀=0, αₖ₊₁=1, αⱼ₋₁<αⱼ. A gene with coordinates (tᵢ,dᵢ) above the reference line is assigned a category of differential expression Cⱼ⁺, if C_{α}⁺<dᵢ≧C_{α}_{ⱼ₊₁}⁺, where C_{α}⁺ is the empirical α-percentile of the distribution of d for genes in the layer Wₜ_{ᵢ}⁺. All genes in Wₜ⁻ under the line are categorized in a similar manner. In fact, as Wₜ depends on t, C_{α}_{ⱼ} is a function of t representing a moving-average estimator of the αⱼ-percentile of the distribution of d given t. The step-functions C_{α}_{₁}(t) cut the plane into 2k+1 percentile bands Bⱼ⁺={0≦t<∞, C_{α}_{ⱼ}⁺<d≦C_{α}_{ⱼ₊₁}⁺} and Bⱼ⁻={0<t<∞, C_{α}_{ⱼ}⁻<d₁≧C_{α}_{ⱼ₊₁}⁻} (the bands B₀⁺ and B₀⁻ are combined into a single one).

To keep the estimation accuracy to a constant, Δ is treated as data-adaptive and such that for any t the layer Wₜ contains approximately the same number of points. A constraint can be also imposed on the maximal bandwidth.

With k=1, the observed gene expression falls into one of the following three categories: “Overexpressed” (the point is in the upper band B₁⁺), “Not differentially expressed” (the point is in the middle band B₀) and “Underexpressed” (the point is in the lower band B₁⁻). With k>1 overexpression and underexpression are represented as a multiple of categories

$$( {X}_{ij} , {Y}_{ij} ) \to \{ \begin{matrix}Overexpressed k & if ( {t}_{ij} , {d}_{ij} ) ∈ {B}_{kj}^{+} \\ … & … \\ Overexpressed l & if ( {t}_{ij} , {d}_{ij} ) ∈ {B}_{lj}^{+} \\ Not diff . expressed & if ( {t}_{ij} , {d}_{ij} ) ∈ {B}_{0 j} \\ Underexpressed l & if ( {t}_{ij} , {d}_{ij} ) ∈ {B}_{lj}^{-} \\ … & … \\ Overexpressed k & if ( {t}_{ij} , {d}_{ij} ) ∈ {B}_{kj}^{-}\end{matrix}$$

One characteristic of this categorical adjustment measure of differential expression is that any rank preserving transformation (possibly dependent on the absolute expression level t) of ideal expression data will be adequately adjusted for.

Under the null hypothesis of no differential expression, genes are expected to show overexpression approximately as often as they show underexpression. In other words, the distribution of a categorical measure of differential expression over a set of slides is symmetric under the null hypothesis.

For a given gene i, introduce the notation: nᵤ⁺=the number of slides where the gene happened to be in category Cᵢ⁺; n₁⁻=the number of slides where the gene happened to be in category Cᵢ⁻; nᵢ⁰=the number of slides where the gene happened to be in category Cᵢ⁰; pᵢ⁺=the probability for the gene of being in category Cᵢ⁺; pᵢ⁻=the probability for the gene of being in category Cᵢ⁻; pᵢ⁰=the probability for the gene of being in category Cᵢ⁰. The total number of slides n=Σᵢ₌₁^{k}(nᵢ⁺+nᵢ⁻)+nᵢ⁰

The null hypothesis that the gene is not differentially expressed can be formulated as pᵢ⁺=pᵢ⁻=pᵢ, i=1, . . . ,k. Under the null hypothesis pᵢ=(nᵢ⁺+n₁⁻)/(2n), pᵤ⁰=n₁⁰/n. Under a saturated model, p₁⁺=nᵢ⁺/n, pᵢ⁻=nᵢ⁻/n, pᵢᵢ⁰=nᵢ⁰/n.

The likelihood ratio statistics can be used to summarize and quantify differential expression over a series of experiments: LR=2Σᵢ₌₁^{k}(nᵢ⁻log(nᵢ⁻)+nᵢ⁺log(nᵢ⁺)−(nᵢ⁻+nᵢ⁺)log(nᵢ⁻+nᵢ⁺)). Under the null hypothesis, LR is asymptotically χ²-distributed with k degrees of freedom. The power of the symmetry-test for differential expression with categorical data can be increased by noting that under the null hypothesis of no difference large over/underexpression should occur less often than a less pronounced deviation. That is, the distribution of the categorical measure of differential expression not only is symmetric and unimodal but it also has monotonically decreasing tails. This suggests an isotonic version of the test for symmetry in order to account for the above mentioned constraint on the corresponding test statistic: p₁⁺=p₁⁻≧p₂⁺=p₂⁻≧ . . . ≧pₖ⁺=pₖ⁻. The maximum likelihood estimates under the ordering restriction may be done by isotonic estimation, for example. See Robertson T. et al., Order Restricted Statistical Inference (Wiley, London, 1988). The asymptotic distribution of the likelihood ratio test statistic is no longer expected to be χ², but rather a mixture of χ² variables with different degrees of freedom. The likelihood ratio statistic computed for each gene may be used to order genes according to their differential expression.

The invention is further described by the following examples, which are illustrative of the invention but do not limit the invention in any manner.

### EXAMPLE 1

### A Source Code Segment Implementing Cross Validated Search of Subsets of Genes Based on Calculation of a Probability Distance Between Vectors

```
unit CrossValThread;
interface
uses
  Classes, Definitions, Matrix, Vector, SysUtils, ComCtrls;
type
  TCrossValThread = class(TThread)
  private
    Dist: VectDistFunc;
    UpdateDist: ThreadUpdateFunc;
    A: TMatrix;
    B: TMatrix;
    size: integer;
    maxit: integer;
    n, k: integer;
    ngenes: integer;
    w1, w2, rangemin, rangemax: double;
    ABss, AAss, BBss: TMatrix;
    ABsame, AAsame, BBsame: TMatrix;
    AAcorr, ABcorr, BBcorr: TMatrix;
    Astand, Bstand: TMatrix; //standardized matrices A and B
    procedure FreeMatrices;
    procedure SetUpdateFunction;
    procedure SetupEuclid;
    procedure SetupKenDist;
    procedure SetupUnsignCorrDist;
    function UpdateHomogeneityDist(ind_in, ind_out: integer;
      SaveChange: boolean):double;
    function UpdateEuclid(X: TMatrix; nx: integer; Y: TMatrix; ny: integer;
      ind_in, ind_out :integer;
      SaveChange: boolean; AuxMat: array of TMatrix): double;
    function UpdateKenDist(X: TMatrix; nx: integer; Y: TMatrix; ny: integer;
      ind_in, ind_out :integer;
      SaveChange: boolean; AuxMat: array of TMatrix): double;
    function UpdateUnsignCorrDist(X: TMatrix; nx: integer; Y: TMatrix; ny: integer;
      ind_in, ind_out :integer;
      SaveChange: boolean; AuxMat: array of TMatrix): double;
    procedure RandomHomoSearch;
  published
    procedure Execute; override;
  public
    indexarr: Tdarray;
    constructor Create(CreateSuspended: boolean; D: VectDistFunc;
        tiss1, tiss2: TMatrix; s, it: integer);
  end;
implementation
uses Math, RandomGen;
{ TCrossValThread }
function Quadrant(x1,x2: double): integer;
var sc1, sc2: double;
begin
    sc1:= (x1−rangemin)/(rangemax−rangemin);
    sc2:= (x2−rangemin)/(rangemax−rangemin);
    Result:= Trunc(kenQ*sc1)*kenQ + Trunc(kenQ*sc2);
end;
constructor TCrossValThread.Create;
var miA, maA, miB, maB: double;
begin
    inherited Create(CreateSuspended);
    Dist:= D;
    A:= tiss1;
    B:= tiss2;
    size:= s;
    maxit:= it;
    n:= tiss1.NrOfRows;
    k:= tiss2.NrOfRows;
    ngenes:= tiss1.NrOfColumns;
    A.MinMax(1,1,ngenes,n,miA,maA);
    B.MinMax(1,1,ngenes,k,miB,maB);
    rangemin:= Min(miA, miB);
    rangemax:= Max(maA, maB);
end;
procedure TCrossValThread.SetUpdateFunction;
begin
    if (@Dist=@Euclid) then
      UpdateDist:= UpdateEuclid
    else if (@Dist=@KenDist) then
      UpdateDist:= UpdateKenDist
    else if (@Dist=@UnsignCorrDist) then
      UpdateDist:= UpdateUnsignCorrDist
    else
      UpdateDist:= UpdateEuclid;
end;
procedure TCrossValThread.Execute;
begin
    SetupEuclid;
    if (@Dist=@KenDist) then
      SetupKenDist
    else if (@Dist=@UnsignCorrDist) then
      SetupUnsignCorrDist
    else if (@Dist <> @Euclid) then begin
      ReturnValue:= 0;
      Exit;
    end;
      SetUpdateFunction;
      RandomHomoSearch;
    indexarr:= Copy(indexarr, 0, size);
    ReturnValue:= Integer(@indexarr);
    FreeMatrices;
end;
procedure TCrossValThread.FreeMatrices;
begin
    FreeAndNil(AAss);
    FreeAndNil(ABss);
    FreeAndNil(BBss);
    FreeAndNil(AAsame);
    FreeAndNil(ABsame);
    FreeAndNil(BBsame);
    FreeAndNil(AAcorr);
    FreeAndNil(ABcorr);
    FreeAndNil(BBcorr);
    FreeAndNil(Astand);
    FreeAndNil(Bstand);
end;
procedure TCrossValThread.RandomHomoSearch;
var i, ranin, ranout, oldmember, newmember: integer;
    maxdist, currdist: double;
begin
    maxdist:= UpdateHomogeneityDist(1, 1, False);
    for i:= 1 to maxit do begin
      if Terminated then begin
        Abort;
      end;
      ranin:= Trunc(Ran0(size)); //# of random member of the Cluster
      ranout:= size + Trunc(Ran0(ngenes-size)); //random gene outside the Cluster
      currdist:= UpdateHomogeneityDist(ranin, ranout, False);
      if (currdist > maxdist) then begin //do the switch
        oldmember:= Trunc(indexarr[ranin]); //the gene to be removed form the cluster
        newmember:= Trunc(indexarr[ranout]); //the gene to be entered into the Cluster
        maxdist:= UpdateHomogeneityDist(ranin, ranout, True); //to update the aux matrices
        indexarr[ranin]:= newmember;
        indexarr[ranout]:= oldmember;
      end
    end;
end;
procedure TCrossValThread.SetupEuclid;
var i, j, g: integer;
begin
    w1:= DefaultKenw1;
    w2:= DefaultKenw2;
    if not Assigned(ABss) then
      ABss:= TMatrix.Create(n,k)
    else
      ABss.Resize(n,k);
    ABss.Fill(0);
    if not Assigned(AAss) then
      AAss:= TMatrix.Create(n,n)
    else
      AAss.Resize(n,n);
    AAss.Fill(0);
    if not Assigned(BBss) then
      BBss:= TMatrix.Create(k,k)
    else
      BBss.Resize(k,k);
    BBss.Fill(0);
    for i:= 1 to n do
      for j:= 1 to k do
        for g:= 0 to size-1 do
          ABss[i,j]:= ABss[i,j] +
            Sqr(A[Trunc(indexarr[g]),i]-B[Trunc(indexarr[g]),j]);
    for i:= 1 to n do
      for j:= 1 to n do
        for g:= 0 to size-1 do
          AAss[i,j]:= AAss[i,j] +
            Sqr(A[Trunc(indexarr[g]),i]-A[Trunc(indexarr[g]),j]);
    for i:= 1 to k do
      for j:= 1 to k do
        for g:= 0 to size-1 do
          BBss[i,j]:= BBss[i,j] +
            Sqr(B[Trunc(indexarr[g]),i]-B[Trunc(indexarr[g]),j]);
end;
function TCrossValThread.UpdateEuclid;
var SSmat: TMatrix;
begin
    SSmat:= AuxMat[0];
    Result:= SSmat[nx, ny]
      − Sqr(X[Trunc(indexarr[ind_in]),nx]−Y[Trunc(indexarr[ind_in]),ny])
      + Sqr(X[Trunc(indexarr[ind_out]),nx]−Y[Trunc(indexarr[ind_out]),ny]);
    if Result < 0 then
      Result:= 0;
    if SaveChange then
      SSmat[nx, ny]:= Result;
    Result:= Sqrt(Result);
end;
procedure TCrossValThread.SetupKenDist;
var i, j, g, g2: integer;
begin
    if (@Dist = @KenDist) then begin
      w2:= DefaultKenw1;
      if not Assigned(ABsame) then
        ABsame:= TMatrix.Create(n,k)
      else
        ABsame.Resize(n,k);
        ABsame.Fill(0);
        if not Assigned(AAsame) then
          AAsame:= TMatrix.Create(n,n)
        else
          AAsame.Resize(n,n);
        AAsame.Fill(0);
        if not Assigned(BBsame) then
          BBsame:= TMatrix.Create(k,k)
        else
          BBsame.Resize(k,k);
        BBsame.Fill(0);
        for i:= 1 to n do
          for j:= 1 to k do
            for g:= 0 to size-1 do
              for g2:= g+1 to size-1 do
                if (Quadrant(A[Trunc(indexarr[g]),i], A[Trunc(indexarr[g2]),i]) <>
                  Quadrant(B[Trunc(indexarr[g]),j], B[Trunc(indexarr[g2]),j]))
                then
                  ABsame[i,j]:= ABsame[i,j] + 1;
        for i:= 1 to n do
          for j:= 1 to n do
            for g:= 0 to size-1 do
              for g2:= g+1 to size-1 do
                if (Quadrant(A[Trunc(indexarr[g]),i], A[Trunc(indexarr[g2]),i]) <>
                  Quadrant(A[Trunc(indexarr[g]),j], A[Trunc(indexarr[g2]),j]))
                then
                  AAsame[i,j]:= AAsame[i,j] + 1;
        for i:= 1 to k do
          for j:= 1 to k do
            for g:= 0 to size-1 do
              for g2:= g+1 to size-1 do
                if (Quadrant(B[Trunc(indexarr[g]),i], B[Trunc(indexarr[g2]),i]) <>
                  Quadrant(B[Trunc(indexarr[g]),j], B[Trunc(indexarr[g2]),j]))
                then
                  BBsame[i,j]:= BBsame[i,j] + 1;
      end;
end;
function TCrossValThread.UpdateKenDist;
var g: integer;
    SSMat, SameMat: TMatrix;
    t1, t2: double;
begin
    SSmat:= AuxMat[0];
    SameMat:= Auxmat[1];
    t1:= SSmat[nx, ny]
      − Sqr(X[Trunc(indexarr[ind_in]),nx]-Y[Trunc(indexarr[ind_in]),ny])
      + Sqr(X[Trunc(indexarr[ind_out]),nx]-Y[Trunc(indexarr[ind_out]),ny]);
    if t1 < 0 then
      t1:= 0;
    t2:= SameMat[nx,ny];
    for g:= 0 to size-1 do begin
      if (g = ind_in) then
        Continue;
      if (Quadrant(X[Trunc(indexarr[g]),nx], X[Trunc(indexarr[ind_in]),nx]) <>
        Quadrant(Y[Trunc(indexarr[g]),ny], Y[Trunc(indexarr[ind_in]),ny])) then
          t2:= t2−1;
      if (Quadrant(X[Trunc(indexarr[g]),nx], X[Trunc(indexarr[ind_out]),nx]) <>
        Quadrant(Y[Trunc(indexarr[g]),ny], Y[Trunc(indexarr[ind_out]),ny])) then
          t2:= t2+1;
    end;
    Result:= w1 * Sqrt(t1)/maxdim + w2 * t2/(maxdim*(maxdim−1)/2);
    if SaveChange then begin
      SSmat[nx, ny]:= t1;
      SameMat[nx,ny]:= t2;
    end;
end;
procedure TCrossValThread.SetupUnsignCorrDist;
var i, j, g, g2: integer;
begin
    w1:= DefaultKenw2;
    w2:= DefaultKenw1;
    if not Assigned(AAcorr) then
      AAcorr:= TMatrix.Create(n,n)
    else
      AAcorr.Resize(n,n);
    AAcorr.Fill(0);
    if not Assigned(ABcorr) then
      ABcorr:= TMatrixCreate(n,k)
    else
      ABcorr.Resize(n,k);
      ABcorr.Fill(0);
    if not Assigned(BBcorr) then
      BBcorr:= TMatrix.Create(k,k)
    else
      BBcorr.Resize(k,k);
    BBcorr.Fill(0);
    if not Assigned(Astand) then
      Astand:= TMatrix.Create(1,1);
    Astand.Clone(A);
    Astand.StandardizeColumns(nil,nil);
    if not Assigned(Bstand) then
      Bstand:= TMatrix.Create(1,1);
    Bstand.Clone(B);
    Bstand.StandardizeColumns(nil,nil);
    for i:= 1 to n do
      for j:= 1 to n do
        for g:= 0 to size-1 do
          for g2:= g+1 to size-1 do
            AAcorr[i,j]:= AAcorr[i,j] + 1 + Max(
              Astand[Trunc(indexarr[g]),i]*Astand[Trunc(indexarr[g2]),i],
              Astand[Trunc(indexarr[g]),j]*Astand[Trunc(indexarr[g2]),j]);
    for i:= 1 to k do
      for j:= 1 to k do
        for g:= 0 to size-1 do
          for g2:= g+1 to size-1 do
            BBcorr[i,j]:= BBcorr[i,j] + 1 + Max(
              −Bstand[Trunc(indexarr[g]),i]*Bstand[Trunc(indexarr[g2]),i],
              −Bstand[Trunc(indexarr[g]),j]*Bstand[Trunc(indexarr[g2]),j]);
      for i:= 1 to n do
        for j:= 1 to k do
          for g:= 0 to size-1 do
            for g2:= g+1 to size-1 do
              ABcorr[i,j]:= ABcorr[i,j] + 1 + Max(
                Astand[Trunc(indexarr[g]),i]*Astand[Trunc(indexarr[g2]),i],
                −Bstand[Trunc(indexarr[g]),j]*Bstand[Trunc(indexarr[g2]),j]);
end;
function TCrossValThread.UpdateUnsignCorrDist;
var g: integer;
    SSMat, CorrMat, Xst, Yst: TMatrix;
    t1, t2: double;
begin
    SSmat:= AuxMat[0];
    CorrMat:= AuxMat[2];
    Xst:= AuxMat[3];
    Yst:= AuxMat[4];
    t1:= SSmat[nx, ny]
      − Sqr(X[Trunc(indexarr[ind_in]),nx]−Y[Trunc(indexarr[ind_in]),ny])
      + Sqr(X[Trunc(indexarr[ind_out]),nx]−Y[Trunc(indexarr[ind_out]),ny]);
    if t1 < 0 then
      t1:= 0;
    t2:= CorrMat[nx,ny];
    for g:= 0 to size-1 do begin
      if (g = ind_in) then
        Continue;
    t2:= t2 − Max(
      Xst[Trunc(indexarr[g]),nx]*Xst[Trunc(indexarr[ind_in],nx],
      Yst[Trunc(indexarr[g]),nx]*Yst[Trunc(indexarr[ind_in]),ny]);
    t2:= t2 + Max(
      Xst[Trunc(indexarr[g]),nx]*Xst[Trunc(indexarr[ind_out]),nx],
      Yst[Trunc(indexarr[g]),nx]*Yst[Trunc(indexarr[ind_out]),ny]);
  end;
  Result:= w1 * Sqrt(t1)/maxdim + w2 * t2/(maxdim*(maxdim-1)/2);
  if SaveChange then begin
    SSmat[nx, ny]:= t1;
    CorrMat[nx,ny]:= t2;
  end;
end;
function TCrossValThread.UpdateHomogeneityDist;
var i, j: integer;
begin
  Result:= 0;
  for i:= 1 to n do
    for j:= 1 to k do
      Result:= Result + 2*UpdateDist(A,i,B,j,ind_in,ind_out,SaveChange,
        [ABss,ABsame,ABcorr,Astand,Bstand])/(n*k);
  for i:= 1 to n do
    for j:= 1 to n do
      Result:= Result − UpdateDist(A,i,A,j,ind_in,ind_out, SaveChange,
        [AAss,AAsame,AAcorr,Astand,Astand])/sqr(n);
  for i:= 1 to k do
    for j:= 1 to k do
      Result:= Result − UpdateDist(B,i,B,J,ind_in,ind_out SaveChange,
        [BBss,BBsame,BBcorr,Bstand,Bstand])/sqr(k);
end;
end.
```

### EXAMPLE 2

### A Source Code Segment Implementing Random Search for Differentially Expressed Subsets of Gene that Calls the Routine in Example 1

```
unit RandSearchThread;
interface
uses
  Classes, Definitions, Matrix, SysUtils;
type
  TRandSearchThread = class(TThread)
  private
    Dist: VectDistFunc;
    A: TMatrix;
    B: TMatrix;
    size, listlength: integer;
    maxit: integer;
    n, k: integer;
    ngenes: integer;
  protected
    procedure GetCrossValResults;
    procedure Execute; override;
  public
    constructor Create(CreateSuspended: boolean D: VectDistFunc;
        tiss1, tiss2: TMatrix; s, it, 1: integer);
  end;
implementation
uses CrossValThread, RandomGen;
var StartSet: Tdarray;
  resultlist: string;
{ TRandSearchThread }
constructor TRandSearchThread.Create;
begin
  inherited Create(CreateSuspended);
  Dist:= D;
  A:= tiss1;
  B:= tiss2;
  size:= s;
  maxit:= it;
  listlength:= 1;
  n:= tiss1.NrOfRows;
  k:= tiss2.NrOfRows;
  ngenes:= tiss1.NrOfColumns;
end;
procedure TRandSearchThread.Execute;
begin
  ReturnValue:= 0;
  GetCross ValResults;
  ReturnValue:= Integer(PChar(resultlist));
end;
procedure TRandSearchThread.GetCrossValResults;
var CrossVals: array of TCrossValThread;
  i, q1, q2, s1, s2, r: integer;
  subMat1, subMat2: array of TMatrix;
  rFreq: TIntMatrix;
  rClust: Tdarray;
function PieceSize1(r: integer): integer;
begin
  if (r<=q1) then
    result:= s1
  else
    Result:= s1+1;
end;
function PieceSum1(r: integer): integer;
begin
  if (r<=q1) then
    result:= r*s1
  else
    Result:= r*s1+(r−q1);
end;
function PieceSize2(r: integer): integer;
begin
  if (r<=q2) then
    result:= s2
  else
    Result:= s2+1;
end;
function PieceSum2(r: integer): integer;
begin
  if (r<=q2) then
    result:= r*s2
  else
    Result:= r*s2+(r−q2);
end;
begin
  SetLength(StartSet, ngenes);
  {generate a random starting cluster and sample orders}
  for i:=0 to ngenes-1 do
    StartSet[i]:= i+1;
  RandomPerm(StartSet);
  SetLength(CrossVals, rFold);
  s1:= n div rFold;
  q1:= rFold − (n mod rFold);
  s2:= k div rFold;
  q2:= rFold − (k mod rFold);
  SetLength(SubMat1, rFold);
  SetLength(SubMat2, rFold);
  rFreq:= TIntMatrix.Create(ngenes,2);
  rFreq.Fill(0);
  for i:= 1 to ngenes do
    rFreq[i,2]:= i;
  try
    for r:= 1 to rFold do begin
      subMat1[r−1]:= TMatrix.Create(ngenes, n-PieceSize1(r));
      subMat2[r−1]:= TMatrix.Create(ngenes, k-PieceSize2(r));
      if (r>1) then begin
        subMat1[r−1].CopyFrom(A, 1, 1, ngenes, PieceSum1(r), 1, 1);
        subMat2[r−1].CopyFrom(B, 1, 1, ngenes, PieceSum2(r), 1, 1);
      end;
      if (r<rFold) then begin
        subMat1[r−1].CopyFrom(A,1,PieceSum1(r+1),ngenes,n,1,
      PieceSum1(r));
        subMat2[r−1].CopyFrom(B,1,PieceSum2(r+1),ngenes,n,1,
        PieceSum2(r));
        end;
      CrossVals[r−1]:= TCrossValThread.Create(True, Dist,
        subMat1[r−1], subMat2[r−1], size, maxit);
      SetLength(CrossVals[r−1].indexarr, ngenes);
      CrossVals[r−1].indexarr:= Copy(Startset, 0, ngenes);
      CrossVals[r−1].Resume;
  end;
  for r:= 1 to rFold do begin
    rClust:= Pdarray(CrossVals[r−1].WaitFor)♯ ♭;
    for i:= 0 to size-1 do
      rFreq[Trunc(rClust[i]),1]:= rFreq[Trunc(rClust[i]),1]+1;
    Finalize(rClust);
  end;
finally
  Finalize(StartSet);
  rFreq.SortCols(1,False,1,1,ngenes,2);
    resultlist:=IntToStr(rFreq[1,2]);
  for i:= 2 to listlength do
    resultlist:= resultlist + ‘, ’ + IntTostr(rFreq[i,2]);
  for r:= 1 to rFold do begin
    subMat1[r−1].Free;
    subMat2[r−1].Free;
    CrossVals[r−1].Free;
  end;
  rFreq.Free;
  end;
end;
end;
```

### EXAMPLE 3

### Cross-Validated Random Search Procedure

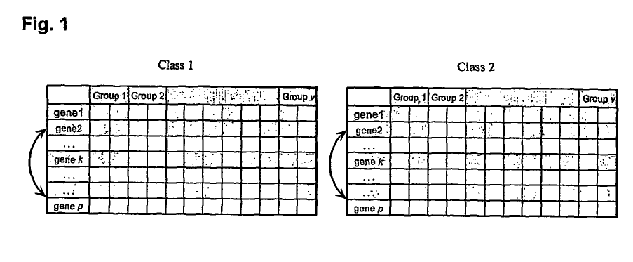

Referring to FIG. 1, suppose there are p genes and n and m independent samples in the two classes respectively, this procedure finds a group of k genes that provides the largest distance between these classes using a v-fold cross-validated search.

1. The samples in both classes are randomly divided into v groups of almost equal size: if nᵥ=Int(n/v), then in Class 1 there will be v-vnᵥ groups of size nᵥ+1 and vnᵥ groups of size nv; Class2 is divided similarly (Groups 1 through v in FIG. 1).

2. Randomly select k genes that will serve as the seed of the random search.

3. For each of the groups do the following:

a. Temporarily discard this group (Group 2 in FIG. 1), that is, consider only the samples not belonging to it.

b. Calculate the distance between the two classes based on the k initially selected genes.

c. Randomly select a gene from the current gene-cluster (gene 2 in FIG. 1), remove it from the cluster and replace it with a gene randomly selected from outside of the cluster.

d. Calculate the distance between the two classes based on the new gene-cluster. If this distance is larger than the previously calculated one, then keep the change, otherwise revert to the previous cluster.

e. Repeat steps c.-d. until convergence. Convergence can be defined in several ways: i. no improvement has been made in a certain number of steps; ii. the (absolute or relative) improvement has been smaller than a specified limit; or iii a predetermined (large) number of steps have been made.

f. Retain the final cluster of genes.

4. After completing the previous step v groups of genes of size k each are obtained. Calculate the frequency of occurrence as a member of the selected group for each gene.

5. The final set of genes can be selected in several ways: i. select the genes with a frequency of occurrence exceeding a preset limit (for example, 0.5 v); ii. select the genes with the k highest frequencies of occurrence; iii. select all the genes that have occurred in at least one of the v clusters.

### EXAMPLE 4

### Classification of AML vs ALL Genes by Cross-Validated Search for the Maximized Distance

A leukemia data set was analyzed; the data set was derived from 27 is ALL (acute lymphoblastic leukemia) and 11 AML (acute myeloid leukemia) samples processed using Affymetrix GeneChip arrays. See Golub et al., Science 1999 286:531-537 (showing that the two classes could be well separated using 10 or more genes as predicators).

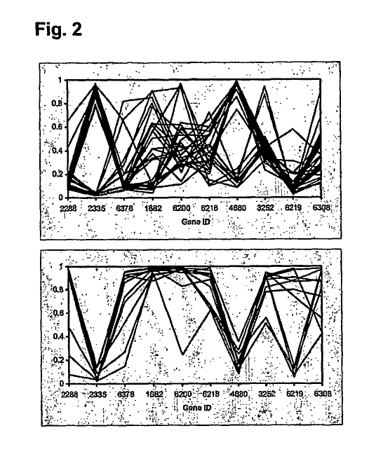

A ten-fold cross-validated search was performed on this data set, for the best subset of genes maximizing the estimated distance D=\sqrt{N(μ,ν)} with the Euclidean distance kernel. The search was repeated 50 times with 10,000 iterations each to find the most differentially expressed cluster of size 10. The procedure was applied to rank-adjusted data. The list of the selected genes is described supra in the brief description of FIG. 2, which shows a line plot of the corresponding expression levels. Three of these genes were also included in the group of 50 predictors by Golub et al. This set of genes provides a 95% cross-validated correct classification rate and the prediction on the test set is perfect with the exception of two samples where a decision is not made due to an extremely low prediction strength (the same is true for genes selected by Golub et al.). The prediction strength was calculated as PS═|D₁−D₂/max(D₁, D₂), where D₁ and D₂ are distances between the sample to be classified and each of the two classes. It measures the confidence level of classifying a given sample.

A noticeable feature of the plot in FIG. 2 is that the ALL samples appear to be divided into two groups. These groups turn out to correspond to the T-cell/B-cell division of the ALL samples. This analysis suggests two genes (# 2335 and # 4680) for discrimination between the groups; they both are well known as markers for T-cell leukemia. It is worth noting that a marginal search would not turn up these genes, because, taken individually, they misclassify B-cell ALL samples but, their sensitivity to T-cell leukemia samples makes them valuable predictors in multivariate classification.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

1. A method for identifying a set of genes from a multiplicity of genes whose expression levels at two states are measured in replicates using one or more probe arrays, thereby generating a plurality of independent measurements of the expression levels, wherein said set is no larger than the plurality, which method comprises:
constructing two random vectors, each corresponding to one of the two states and comprising the expression levels of a group of genes, wherein the group is a random subset of said multiplicity;
calculating probability distance(s) between said two random vectors using a probability distance formula; and
determining an advantageously large probability distance between said two random vectors;
wherein the group of genes which constitute the two random vectors giving rise to the advantageously large probability distance is the set of genes identified.

2. The method of claim 1, wherein said states are selected from the group consisting of biological states, physiological states, pathological states, diagnostic and prognostic states.

3. A method for identifying a set of genes from a multiplicity of genes whose expression levels in two or more cell types or tissues are measured in replicates using one or more nucleotide arrays, thereby generating a plurality of independent measurements of the expression levels, wherein said set is no larger than the plurality, which method comprises:
constructing two random vectors, each corresponding to one of the two cell types or tissues and comprising the expression levels of a group of genes, wherein the group is a random subset of said multiplicity;
calculating probability distance(s) between said two random vectors using a probability distance formula; and
determining an advantageously large probability distance between said two random vectors;
wherein the group of genes which constitute the two random vectors giving rise to the advantageously large probability distance is the set of genes identified.

4. A method for identifying a set of genes from a multiplicity of genes whose expression levels in two or more tissues are measured in replicates using one or more nucleotide arrays, thereby generating a plurality of independent measurements of the expression levels, wherein said set is no larger than the plurality, which method comprises:
constructing two random vectors, each corresponding to one of the two tissues and comprising the expression levels of a group of genes, wherein the group is a random subset of said multiplicity;
calculating probability distance(s) between said two random vectors using a probability distance formula; and
determining an advantageously large probability distance between said two random vectors;
wherein the group of genes which constitute the two random vectors giving rise to the advantageously large probability distance is the set of genes identified.

5. The method of claim 3 or claim 4, wherein said tissues are selected from the group consisting of normal lung tissues, abnormal lung tissues, cancer lung tissues, normal heart tissues, pathological heart tissues, normal and abnormal colon tissues, normal and abnormal renal tissues, normal and abnormal prostate tissues, and normal and abnormal breast tissues.

6. A method for identifying a set of genes from a multiplicity of genes whose expression levels in two or more types of cells are measured in replicates using one or more nucleotide arrays, thereby generating a plurality of independent measurements of the expression levels, wherein said set is no larger than the plurality, which method comprises:
constructing two random vectors, each corresponding to one of the two types of cells and comprising the expression levels of a group of genes, wherein the group is a random subset of said multiplicity;
calculating probability distance(s) between said two random vectors using a probability distance formula; and
determining an advantageously large probability distance between said two random vectors;
wherein the group of genes which constitute the two random vectors giving rise to the advantageously large probability distance is the set of genes identified.

7. The method of claim 3, wherein said types of cells are selected from the group consisting of normal lung cells, cancer lung cells, normal heart cells, pathological heart cells, normal and abnormal colon cells, normal and abnormal renal cells, normal and abnormal prostate cells, and normal and abnormal breast cells.

8. The method of claim 3, wherein said type of cells are selected from the group consisting of cultured cells and cells isolated from an organism.

9. The method of any claim 1, wherein the advantageously large distance is a maximal probability distance taken over the plurality of independent measurements.

10. The method of claim 1 wherein the probe arrays are nucleotide arrays.

11. The method of claim 10 wherein said nucleotide arrays are selected from the group consisting of spotted arrays and in situ synthesized arrays.

12. The method of claim 1, wherein the probability distance is selected from the group consisting of the Mahalanobis distance and the Bhattacharya distance.

13. The method of claim 1, wherein the probability distance formula is N(μ,ν)=2∫ₒ_{^{d}}∫ₒ_{^{d}}L(x, y)dμ(x)d ν(w)−∫ₒ_{^{d}}∫ₒ_{^{d}}L(x, y)dμ(x)dμ(y)−∫ₒ_{^{d}}∫ₒ_{^{d}}L(x, y)dν(x)dν(w) where μ and ν are two probability measures defined on the Euclidean space, and L(x,y) is a strictly negative definite kernel.

14. The method of claim 13, wherein the negative definite kernel is combined with the Euclidean distance between x and y to form a composite kernel function.

15. The method of claim 13, wherein the negative definite kernel is based on the correlation coefficient and is capable of detecting differences in correlation between the two random vectors.

16. The method of any of claim 1, wherein the expression levels are adjusted to their corresponding fractional ranks as compared to one another and thereafter used to construct said random vectors.

17. The method of claim 1, wherein each of the expression levels is adjusted to a corresponding categorical descriptor of the extent of over or under expression and thereafter used to construct said random vectors.

18. The method of claim 4, wherein said tissues are selected from the group consisting of normal lung tissues, abnormal lung tissues, cancer lung tissues, normal heart tissues, pathological heart tissues, normal and abnormal colon tissues, normal and abnormal renal tissues, normal and abnormal prostate tissues, and normal and abnormal breast tissues.

19. The method of claim 6, wherein said types of cells are selected from the group consisting of normal lung cells, cancer lung cells, normal heart cells, pathological heart cells, normal and abnormal colon cells, normal and abnormal renal cells, normal and abnormal prostate cells, and normal and abnormal breast cells.

20. The method of claim 3, wherein the advantageously large distance is a maximal probability distance taken over the plurality of independent measurements.

21. The method of claim 4, wherein the advantageously large distance is a maximal probability distance taken over the plurality of independent measurements.

22. The method of claim 6, wherein the advantageously large distance is a maximal probability distance taken over the plurality of independent measurements.
